(19) United States
(12) Patent Application Publication
(10) Pub. No.: US 2025/0235584 A1
(43) Pub. Date: Jul. 24, 2025

(54) RECONSTRUCTIVE COMPOSITIONS AND USES THEREOF

(71) Applicant: The Feinstein Institutes for Medical Research, Manhasset, NY (US)

(72) Inventors: Neil Tanna, Manhasset, NY (US); Mark L. Smith, Huntington, NY (US)

(21) Appl. No.: 19/024,775

(22) Filed: Jan. 16, 2025

Related U.S. Application Data
(60) Provisional application No. 63/622,170, filed on Jan. 18, 2024.

(51) Int. Cl. A61L 27/36; A61F 2/12

(52) U.S. Cl. CPC A61L 27/3695; A61F 2/12; A61L 27/362; A61L 2430/04; A61L 2430/34

(57) ABSTRACT

The present disclosure relates to novel pharmaceutical compositions and their use in general tissue reconstructive and augmentative methods. The pharmaceutical compositions comprise multilayered acellular matrices, such as dermal matrices (ADMs), of two or more layers. The compositions may be used in any general tissue reconstructive or augmentative medical procedure where the goal is to increase the volume in the area of the body in which the multilayered ADM compositions are introduced.

### FIELD OF THE INVENTION

The present disclosure relates to novel pharmaceutical compositions and their use in general tissue reconstructive and augmentative methods. The pharmaceutical compositions comprise multilayered acellular matrices, such as dermal matrices (ADMs), of two or more layers. The compositions may be used in any general tissue reconstructive or augmentative medical procedure where the goal is to increase the volume in the area of the body in which the multilayered ADM compositions are introduced.

### BACKGROUND OF THE INVENTION

Soft tissue losses from tumor removal, trauma, aging, or congenital malformation affect millions of people each year. Such losses of tissue lead to major functional and aesthetic disorders that are difficult to treat by conventional means. For example, hundreds of thousands of partial and total mastectomies are performed in the United States each year, leading to disfiguring breast deformities from the loss of breast soft tissue.

Existing options for soft tissue restoration have significant drawbacks. Autologous tissue flaps require moving soft tissues from one part of the body to another in lengthy surgical procedures that leave donor-site defects. Other therapies, such as prosthetic implants, involve complications such as infection, malposition, extrusion, capsular contracture, rupture and need for surveillance and possible replacement. Existing strategies to promote tissue in-growth are also inadequate for the treatment of soft tissue defects. Current acellular matrices result in flat sheets of tissue rather than the soft, three-dimensional tissue required for ideal reconstructions. Finally, while fat grafting can restore soft tissue defects, its use is impeded by variable graft survival, limited volumes of restoration, and a finite donor volume.

Accordingly, there is need for novel compositions and methods for augmentation of soft tissue. The present disclosure provides novel biocompatible, implantable materials for use in soft tissue augmentation without the defects or limitations of currently available therapies.

### SUMMARY OF THE INVENTION

The present disclosure relates to novel pharmaceutical compositions and their use in general tissue reconstructive and augmentative methods. The pharmaceutical compositions comprise multilayered acellular matrices of two or more layers wherein said acellular matrices may be derived from a number of different tissue types. In a non-limiting embodiment, the multilayered acellular matrices are multilayered acellular dermal matrices (ADMs). Although the disclosure below is described for use of ADMs, it is to be understood that acellular matrices derived from tissue, other than dermal tissue, may also be utilized equally as well in the compositions and methods provided herein.

The compositions may be used in any general tissue reconstructive medical procedures, e.g., for repair, where the goal is to increase the tissue volume in the area of the body in which the multilayered ADMs are introduced. Said compositions are also referred to herein as multilayered ADM implants. The compositions may also be used in any general tissue augmentation medical procedure where the goal is to increase the volume in a desired area of the body in which the multilayered ADM implants are introduced.

In an embodiment, a pharmaceutical composition is provided comprised of multilayered acellular dermal matrices (ADMs) wherein the multilayered ADM comprises two or more layers. The multilayered ADM may be derived from a human or animal source. In an embodiment, the compositions comprise a sheet of ADM that is folded on itself at least once. Alternatively, the multilayered ADM composition is one wherein two or more sheets of ADM are layered on top of each other. In another aspect, the ADM is rolled to form multilayers. The multilayered ADM may be designed into any three-dimensional form, for example, such as in the shape of a mound. An ADM composition for implantation as provided herein may further comprise a combination of one or more multilayered ADMs to form said implant, e.g., a multilayered ADM encased in a second multilayer ADM. The multilayered ADM compositions may further comprise a bioagent such as a therapeutic agent, a peptide or a nucleic acid.

The pharmaceutical compositions provided herein comprise a multilayered ADM composition, for augmenting soft tissue in a subject undergoing a surgical procedure, wherein the multilayered ADM is in an amount effective to provide for the retention of a tissue shape when administered to the subject. Said pharmaceutical compositions may further include pharmaceutical carriers such as those well known to those of skill in the art.

Further provided are manufacturing methods for production of multilayered ADM products for use in repairing or filling soft tissues. In one aspect, the present disclosure provides the manufactured products for use by surgeons in the repair or filling of soft tissues. In certain embodiments, the multilayered ADM is prepared until the desired volume and shape, for a desired purpose, is achieved. For breast reconstruction, or augmentation, the multilayered ADM is shaped into a mound-like shape with the desired volume. In one embodiment, the multilayered ADM implants are configured/designed to produce a range of different specific breast shapes and sizes.

The present disclosure provides novel methods for restoration of soft tissue loss with the use of the provided multilayered ADM compositions. Specifically, disclosed herein is a method of treating a soft tissue deformity or deficit, comprising (i) identifying an anatomic site within a soft tissue in need of restoration; (ii) selecting a multilayered ADM composition; (iii) implanting or injecting the multilayered ADM composition in or proximate to the soft tissue; and (iv) securing at least a portion of the multilayered ADM composition in or near the soft tissue. In non-limiting embodiments the use of such soft tissue restoration is desired where there have been soft tissue losses from surgery, tumor removal, trauma, aging, or congenital malformation. In some embodiments, the soft tissue is facial, abdominal, trunk, extremity, ear, nose or penile soft tissue. In another embodiment, the tissue is breast tissue.

The present disclosure also provides novel methods for augmentation, i.e., enhancement, of soft tissue deficits with the use of the provided multilayered ADM compositions. Specifically, disclosed herein is a method of treating a soft tissue deformities or deficits, comprising (i) identifying an anatomic site within a soft tissue in need of augmentation; (ii) selecting a multilayered ADM composition; (iii) implanting or injecting the multilayered ADM composition in or proximate to the soft tissue; and (iv) securing at least a portion of the multilayered ADM composition in or near the soft tissue, In non-limiting embodiments the use of such soft tissue augmentation is desired where there are soft tissue deficits. In some embodiments, the soft tissue is facial, abdominal, trunk, extremity, ear, nose or penile soft tissue. In one aspect, the multilayered ADM compositions are used for cosmetic applications. In another embodiment, the tissue is breast tissue.

The multilayered ADM compositions described herein are suitable for use in breast surgery, and more particularly for breast augmentation and reconstruction procedures. Such reconstruction procedures can be performed after removal of a breast implant or breast reconstruction procedures after, for example, a mastectomy. Such augmentation procedures may include cosmetic augmentation of breast tissue.

In one embodiment, a method of implanting the implants comprises at least the steps of: (i) making at least one incision to access the patient's breast tissue, (ii) separating the skin and subcutaneous fascia of the breast mound from the breast, (iii) placing the implant on the breast mound of the breast, (iv) securing the implant to the tissue surrounding the breast mound of the breast and (v) closing the incisions in the breast.

In addition to the use of multilayered ADM compositions singularly for breast reconstruction, in a non-limiting embodiment, a hybrid method for breast reconstruction is provided comprising the steps of (i) removing a sample of flap tissue from the subject; (ii) forming a multilayered ADM composition; and (iii) introduction of the flap and multilayered ADM composition to at least one breast of a subject. In such a hybrid method, the flap is an autologous flap of fat, placed superficial to a multilayered ADM composition. The flap tissue may be selected from the group consisting of: a transverse rectus abdominis muscle (TRAM) flap; an abdomen deep inferior epigastric perforator (DIEP) flap; an abdomen superficial inferior epigastric artery (SIEA) flap; a back latissimus dorsi flap; a gluteal flap; an inner thigh (TUG) flap; a lumbar artery perforator (LAP) flap and an profunda artery perforator (PAP) flap. In such a hybrid method the subject maybe a woman who has undergone a lumpectomy, quadrantectomy, a total or partial mastectomy, and/or radiation therapy.

Alternatively, the hybrid method may be used for cosmetic improvement of the breast wherein the goal is to change the size, shape, and contour of the subject's breast. Said method comprises introduction of the multilayered ADM composition to at least one breast of a subject. The present invention further provides kits comprising multilayered ADM pharmaceutical compositions and instructions for their use. Said pharmaceutical compositions comprise a multilayered ADM composition, for reconstruction or augmentation of soft tissue in a subject undergoing a surgical procedure, wherein the multilayered ADM is in an effective amount to provide for the retention of a tissue shape when administered to the subject. Said kits may further include pharmaceutical carriers such as those well known to those of skill in the art.

In one aspect, kits are provided comprising multilayered ADM products for breast reconstructive or augmentative procedures. In such instances, for breast reconstruction or augmentation, the kits are designed to include two or more of the multilayered ADM compositions molded into a mound-like shape with the desired volume and shape. The mound shaped multilayered ADM for implantation can be configured or designed to produce a range of different specific breast shapes and sizes to be included in the kits. The availability of said kits provides a means for the surgeon to choose a specific product, e.g., to personalize the selection of the product, to produce a breast implant with a highly desirable appearance, e.g., shape and volume, for the patient.

### BRIEF DESCRIPTION OF THE DRAWINGS

FIG. 1. The layers within the multilayered ADM composition provide thickness to the composition which yields increased volume.

FIG. 2. The multilayered ADM composition can be configured in various designs. Pictured here is a design configured to serve a similar purpose as a traditional breast implant (but to a lesser volume).

FIG. 3. The multilayered ADM composition can be designed in various shapes and sizes, yielding varying weights.

FIG. 4. In the HyPAD (hybrid flap, prepectoral acellular dermal matrix) technique, the multilayered ADM serves the similar purpose as a small implant, but to a lesser degree, providing soft tissue augmentation of the flap and core projection. Various angles (FIG. 4A-D) of the multilayered ADM construct are shown. The multilayered ADM is compared to a silicone breast prosthesis (FIG. 4E).

FIG. 5. Possible donor sites for autologous reconstruction. The most common source of autologous reconstruction is the DIEP flap. Alternative donor sites include the lumbar artery perforator (LAP) flap, pro-funda artery perforator (PAP) flap, TUG flap, and the gluteal artery perforator (GAP) flap. Other sites may be similarly employed.

FIG. 6. DIEP flap breast reconstruction.

FIG. 7. The HyFIL® (hybrid flap, implant, lipofilling) technique combines flap, pre-pectoral implant, and fat transfer into one integrated procedure.

FIG. 8. The HyPAD™ (hybrid flap, prepectoral acellular dermal matrix) technique combines flap reconstruction with multilayered prepectoral ADM to provide additional volume augmentation and core projection. The HyPAD technique is similar to the HyFIL® technique, except the implant is replaced with a multilayered ADM composition.

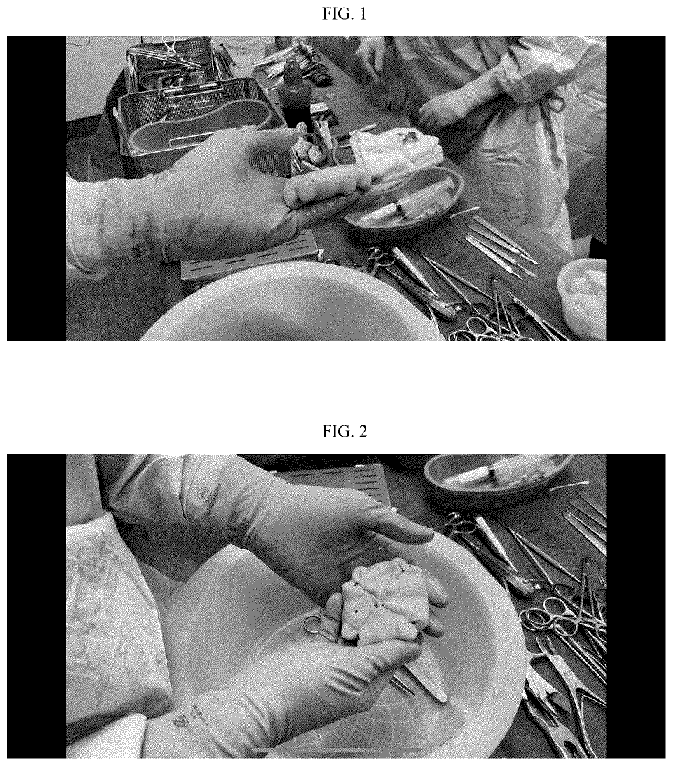

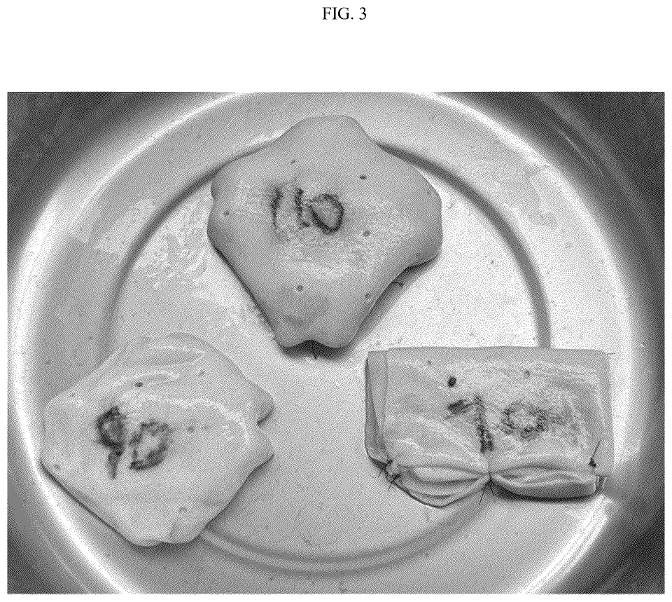

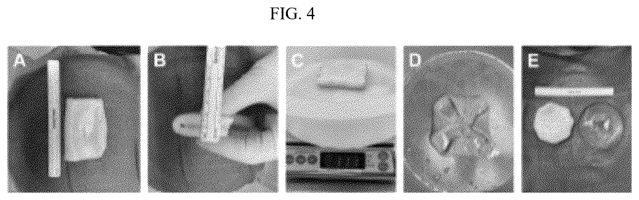

### DETAILED DESCRIPTION OF THE INVENTION

The following is a detailed description of the novel compositions and methods for soft tissue augmentation. Unless otherwise defined, all technical and scientific terms used herein have the same meaning as commonly understood by one of ordinary skill in the art to which this invention belongs. The terminology used in the present disclosure is for describing particular embodiments only and is not intended to be limiting by the disclosure. All publications mentioned herein are incorporated herein by reference to disclose and described the methods and/or materials in connection with which the publications are cited.

The present disclosure relates to novel reconstructive pharmaceutical compositions for use in general soft tissue restorative methods. The reconstructive pharmaceutical compositions comprise multilayered acellular dermal matrices (ADMs) of two or more layers. Accordingly, the present disclosure relates to a method of fabricating a composition for use in reconstruction where the composition comprises multilayered acellular dermal matrices (ADMs) of two or more layers. The compositions may be used in any general tissue reconstructive medical procedures where the goal is to increase, or restore, the volume in the area of the body in which the multilayered ADMs are introduced. More specifically, the disclosure provides methods for repairing or reconstructing a soft tissue injury using a composition comprising the multilayered ADMs disclosed herein.

The present disclosure further relates to novel soft tissue augmentation pharmaceutical compositions for use in general soft tissue volume enhancing methods. Said pharmaceutical compositions comprise multilayered ADMs of two or more layers. Accordingly, the present disclosure relates to a method of fabricating a composition for use in tissue augmentation wherein the composition comprises multilayered ADMs of two or more layers. The compositions may be used in any general tissue augmentation medical procedures where the goal is to increase the tissue volume in the area of the body in which the multilayered ADMs are introduced. More specifically, the disclosure provides methods for increasing tissue volume using a composition comprising the multilayered ADMs disclosed herein. For example, in a non-limiting embodiment, the provided method may be utilized cosmetically to change the size, shape, and contour of a subject's breast. More specifically, methods also include, but are not limited to, breast augmentation wherein the multilayered ADMs are used in place of traditional saline or silicone breast implants. In general, the ADMs for use in the present disclosure are composed of allograft tissue that is processed to remove epidermal and dermal cells while preserving the remaining bioactive dermal matrix, While not being bound to any particular theory, the ADMs function by providing a bioactive matrix consisting of collagens, elastin, blood vessel channels, and bioactive proteins that support revascularization, cell repopulation, and tissue remodeling.

Multiple benefits are associated with the use of multilayered ADM implants. For example, said implants maintain their implant position, have capsular stability, are non-inflammatory and long lasting. The implant does not need to be replaced, thereby reducing the need for re-operation, which decreases risks, expense and potential morbidity for patients in the future. One particular noteworthy feature of ADM compositions is related to immunology as said implants are found not to be associated with autoimmune illnesses or secondary malignancies that have been reported with breast implants. Additionally, said ADM based implants can be constructed in various configurations or designs to match the intended volume of the recipient site thereby providing a reliable volume. In contrast, fat transfer or fat grafting has variable viability or successful establishment at the recipient site.

The success of ADM has been reported to depend on the proper integration with the surrounding tissues. Failure to integrate effectively can result in complications like poor wound healing or graft failure. Therefore, it is a surprising result that the multilayered ADMs, whose inner aspects would not be in direct contact with surrounding tissue, function for their intended use for support of revascularization, cell repopulation, and tissue remodeling thereby increasing their stability over long periods of time,

In comparison with traditional saline or silicone breast implants, through the use of ADM based implants, one can avoid implant rupture, the need for breast implant imaging (ultrasound or MRI) to monitor for rupture, capsular contracture (hardening), implant malposition and breast implant illness.

The present disclosure provides multilayered ADM compositions and methods of use. Accordingly, in some non-limiting embodiments, a soft tissue reconstruction or augmentation product comprising multilayered ADM is provided. ADM sheets used in the formation of multilayered ADM, are commercially available and are typically developed from human, porcine, or bovine skin. Commercially available ADM sheets are generally prepared in a process wherein the cell components in the skin are completely removed, leaving only extracellular matrix proteins and collagen in the dermis. One noteworthy feature of the ADM is that the remaining extracellular matrix proteins and collagen are non-immunogenic, as the human, porcine and bovine antigenic components are removed. Therefore, there is an absence of a rejection reaction after transplantation.

Commercially available ADM include those available from AlloDerm® (LifeCell Corp., Bridgewater, NJ, USA; Human, non-cross-linked); AlloMax® (CR Bard/Davol Inc., Cranston, RI, USA; Human, non-cross-linked); DermACELL® (LifeNet Health Inc., Virginia Beach, VA, USA; Human, non-cross-linked); FlexHD® (Ethicon, Inc., Somerville, NJ, USA; Human, non-cross-linked); Cortiva® (RTI Surgical, Alachua, FL, USA; Human, non-cross-linked); Integra™ (Integra Life Sciences, Princeton, NJ, USA; Bovine, cross-linked); MatriDerm® (Dr Suwelack AG, Billerbeck, Germany; Bovine, non-cross-linked); SurgiMend® (Integra Life Sciences, Princeton, NJ, USA; Bovine, non-cross-linked); Strattice® (Allergan, Madison, NJ, USA; Porcine, non-cross-linked); Permacol™ (Medtronic, Minneapolis, MN, USA; Porcine, cross-linked) and CollaMend™ (CR Bard/Davol Inc., Cranston, RI, USA; Porcine, cross-linked). In another embodiment, other collagen matrices may be used such as those derived from acellular intestinal submucosa. Such matrices are available commercially (such as from Surgisis, Biodesign or CorMatrix).

The unique three-dimensional structure of the multilayered ADM compositions disclosed herein provide a three-dimensional frame for growth and metabolism for tissue cells, and extracellular matrix proteins, which can promote the attachment and proliferation of cells, so that incorporation of the ADM is possible in areas where it contacts the living tissue. Accordingly, the multilayered ADM compositions disclosed herein have excellent biocompatibility and can be used advantageously as novel material for tissue reconstruction and augmentation. In an embodiment, the multilayered compositions described herein can be produced from a variety of different human or animal tissues including human, porcine, ovine, bovine, or other animal tissues. In a specific non-limiting embodiment, the multilayered compositions are dermal ADM compositions.

The present disclosure provides compositions comprising ADM material, e.g., sheets of ADM, that have been multilayered prior to introduction into the soft tissue area in need of restoration and/or augmentation. In an embodiment, the multilayered ADMs for implantation may have a variety of different sizes, shapes and thickness levels. In a specific embodiment, the multilayered ADM comprises at least two or more layers of ADM. The ADM sheets may be folded 2, 3, 4 or additional multiple times onto itself to create a geometric shaped pad that is multiple layers thick. In an embodiment the pad can be any geometric shape such as rectangular or square. In some embodiments the ADM multilayered sheets are spherical, circular or conical in shape. In an embodiment a more complex folding pattern is used to create a rounder three-dimensional shaped pad of ADM, e.g., a mound. These pads can typically add a 1.5 to 2 cm of central projection to the reconstructed breast mound. In yet another embodiment, rather than folding, the ADM sheets can be stacked one on top of the other. In another embodiment, the ADM sheets can be rolled into a multilayered ADM composition. In yet another embodiment, a folded ADM composition may be positioned within a folded ADM composition, e.g., encased. Such encasements may be performed multiple times until the desired volume of the ADM composition is arrived at

In an embodiment, the multilayered ADMs disclosed herein may be modified so as to contain a bioactive agent and thereby act as an active agent delivery system when applied to a body surface (e.g., a site of tissue reconstruction or augmentation). Such bioactive agents may be incorporated into the stacked ADMs during manufacturing or may be mixed in at the time of implantation. Bioctive agents include, for example, small molecules, nucleic acids, polypeptides, soft and hard tissue inductive growth factors (such as fibroblast growth factor (FGF), epidermal growth factor (EGF), platelet derived growth factor (PDF), etc.) and therapeutic reagents. Nucleic acid molecules may include DNA, RNA, siRNA, miRNA, antisense or aptamers.

Other bioactive agents that can be incorporated into the ADM based implants include antimicrobial agents, in particular antibiotics, oncological agents, anti-scarring agents, anti-inflammatory agents, anesthetics, small molecule drugs, anti-angiogenic and pro-angiogenic factors. factors, immunomodulatory agents and blood clotting agents. Suitable antimicrobial agents include: bacitracin, biguanide, triclosan, gentamicin, minocycline, rifampicin, vancomycin, cephalosporins, copper, zinc, silver and gold.

In a further aspect the present disclosure provides a multilayered ADM composition for retaining tissue shape in a subject undergoing a surgical procedure, that includes the multilayered ADM in an amount effective to provide for the retention of a desired tissue shape when administered to the subject. The present disclosure also provides use of the multilayered ADM compositions in preparing products for repairing or filling soft tissues. The use may specifically be, but is not limited to, applications such as breast remodeling and the like. The multilayered ADM compositions provide herein possess the tissue engineering characteristics of no immunological rejection, lack of volume loss or leakage, elasticity and soft texture making them ideal devices as applied to wound repair and tissue defect filling. Multilayered ADM compositions, when implanted on or into subdermal tissue mammary tissue, or other tissue, may be selected to allow regeneration of the tissue without excessive fibrosis, capsular contracture, or scar formation.

The present disclosure also relates to methods for manufacture of stacked ADM implants. To carry out the proper design and manufacture of the stacked ADM compositions for a particular use, parameters such as size and shape are selectively established to obtain the targeted implant characteristics for a particular tissue site. The composition is preferably prepared by starting with a single layer of ADM material and forming a multilayer ADM composition through stacking, folding, rolling or any other method that results in a multilayer ADM composition. In embodiments, the implants for manufacture can be configured/designed to produce a range of different specific shapes and sizes depending on the end use. In certain embodiments, the multilayered ADM is prepared until the desired volume and shape, for a desired purpose, is achieved. For breast reconstruction, or augmentation, the multilayered composition is shaped into a mound-like shape with the desired volume.

In one embodiment, a multilayered ADM implant is designed with a desired shape that produces specific volumetric proportions of soft tissue in the breast. In further aspects, the implant has a predetermined three-dimensional shape that can be implanted subcutaneously to cover the entire breast, between the skin and the mammary mound of the breast. The multilayered ADM implant allows the surgeon to easily control the volumetric proportions of the breast, the degree of protrusion of the breast from the chest wall, and the curvatures of the breast. In embodiments, the implant has a full contour design and provides a means for the surgeon to produce a breast with highly desirable shapes and volumes. In embodiments, the implants are configured/designed to produce a specific breast shape breast to be reshaped in a single procedure.

In one embodiment, a plurality of implant sizes can be provided. The various sizes and shapes of the multilayered ADM implants can be made from varying thicknesses of ADM. For example, the starting ADM material can be of various thicknesses, e.g., thin, medium, thick or extra thick profiles.

In one aspect, the multilayered ADM breast implants are designed so that when manufactured they are three-dimensional. Its shape allows the surgeon to contour the breast and control the volumes of the upper and lower poles. Implants have volumetric dimensions that produce specific breast shapes when implanted. Specifically, the volumetric dimensions of the implant sculpt the breast so that the relationship between the upper pole volume (UPV) and the lower pole volume (LPV) is predefined by the implant.

In one embodiment, the ADM implant has properties that allow it to be delivered through a small incision. The ADM implant can, for example, be designed so that it can be rolled or folded to allow placement through a small incision. This minimally invasive approach can reduce patient morbidity, scarring, and the potential for infection. In another embodiment, the implant has a three-dimensional shape that allows it to assume its original three-dimensional shape without assistance after being placed through an incision. For example, the implant can be temporarily deformed by rolling it into a small diameter cylindrical shape, placing it with an inserter, and then allowing it to return to its original three-dimensional shape without live assistance.

In some embodiments, the multilayered ADM compositions are inserted into a human patient, an animal patient, or a laboratory animal. The provided compositions can be injected intradermally, subcutaneously, sub-dermally, or surgically implanted. Accordingly, the stacked ADM is formulated for intradermal, subcutaneously, subdermal or implanted administration

In some embodiments, the multilayered ADM product is implanted across a tissue defect, such as a soft tissue defect. An additional aspect provides a method for resolving a tissue defect resulting from a trauma or surgical intervention or a method for reducing or reversing a tissue defect resulting from an aging-associated disease, disorder or condition, the methods involving the implantation multilayered ADM compositions.

In non-limiting embodiments the use of such soft tissue restoration is desired where there have been soft tissue losses from surgery, tumor removal, trauma, aging, or congenital malformation. In some embodiments, the soft tissue is facial, abdominal, trunk, extremity, ear, nose or penile soft tissue. In another embodiment, the tissue is breast tissue. Such methods include, but are not limited to, breast reconstruction, more particularly, to techniques for breast reconstruction that rely on the use of the multilayered ADM compositions. The method may be used for the reconstruction of the breast after lumpectomy or after a partial or total mastectomy. The method can also be applied to other organs and parts of the body that have been lost to scarring, radiation, disease or aging and that could benefit from soft tissue augmentation.

In one aspect, a method is provided for augmenting, i.e., enhancing, a soft tissue deficit, the methods involving the implantation of multilayered ADM compositions. In non-limiting embodiments the use of such soft tissue augmentation is desired where there are soft tissue deficits in certain anatomical areas of the body. In some embodiments, the soft tissue is facial, abdominal, trunk, extremity, ear, nose or penile soft tissue. In another embodiment, the tissue is breast tissue. Such methods include, but are not limited to, breast augmentation, as the multilayered ADM implant can be used in lieu of a traditional saline or silicone breast implant.

The present disclosure provides novel methods for restoration of soft tissue loss with the use of the provided multilayered ADM composition. Specifically, disclosed herein is a method of treating a soft tissue deformity or deficit, comprising (i) identifying an anatomic site within a soft tissue in need of restoration; (ii) selecting a multilayered ADM composition; (iii) implanting or injecting the multilayered ADM composition in or proximate to the soft tissue; and (iv) securing at least a portion of the multilayered ADM composition in or near the soft tissue. In non-limiting embodiments the use of such soft tissue restoration is desired where there have been soft tissue losses from surgery, tumor removal, trauma, aging, or congenital malformation. In some embodiments, the soft tissue is facial, abdominal, trunk, extremity, ear, nose or penile soft tissue. In another embodiment, the tissue is breast tissue.

The present disclosure also provides novel methods for augmentation, i.e., enhancement, of soft tissue deficits with the use of the provided multilayered ADM composition. Specifically, disclosed herein is a method of treating a soft tissue deformities of deficits, comprising (i) identifying an anatomic site within a soft tissue in need of augmentation; (ii) selecting a multilayered ADM composition; (iii) implanting or injecting the multilayered ADM composition in or proximate to the soft tissue; and (iv) securing at least a portion of the multilayered ADM composition in or near the soft tissue. In non-limiting embodiments the use of such soft tissue augmentation is desired where there are soft tissue deficits. In some embodiments, the soft tissue is facial, abdominal, trunk, extremity, ear, nose or penile soft tissue. In another embodiment, the tissue is breast tissue.

The multilayered ADM implants described herein are most suitable for use in breast surgery, and more particularly for breast augmentation procedures. However, implants can also be used in other procedures, such as reconstruction procedures after removal of a breast implant and breast reconstruction procedures after a mastectomy. In one embodiment, a method of implanting the multilayered ADM implants comprises at least the steps of: (i) making at least one incision to access the patient's breast tissue, (ii) creating a plane to receive the ADM implant below the breast mount (subglandular) or below the pectoralis chest muscle (subpectoral) (iii) placing the ADM implanting the respective plane, (iv) securing the ADM implant to the tissue surrounding and (v) closing the incisions in the breast.

In a specific embodiment, a procedure is provided that combines surgical flap reconstruction with the use of multilayered ADM compositions, without the use of an implant, thereby allowing for additional core projection without any implant-associated risks. This method is distinguished from previously disclosed methods by the use of surgical flap reconstruction in conjunction with multilayered ADMs. Accordingly, the disclosed method specifically comprises the steps (i) removing a sample of flap tissue from the subject; (ii) forming multilayered ADM; and (iii) introduction of the flap and multilayered ADM to at least one breast of a subject. The subject to be treated may be a human or more particularly a female human. The method disclosed herein may be used in the reconstruction of the breast mound after lumpectomy, quadrantectomy, mastectomy, and/or radiation therapy.

In another aspect, an article of manufacture (e.g., a kit) containing materials useful for restoration or augmentation of soft tissue as described above is provided. In an embodiment, the kit comprises the necessary component of multilayered ADMs and instructions for their use. The article of manufacture comprises a container and a label or package insert on or associated with the container. The container holds a composition which is by itself or combined with another composition effective for restoring or augmenting soft tissue and may have a sterile access port.

In one aspect, the kit may contain, in addition to the standard sheet of ADM, a stencil, a diagram, or an imprint on the ADM itself, that allows for cutting, folding and creating varying sized ADM constructs for implantation. An instruction guide can be included for how to carry out the implantation methods described herein.

The label or package insert indicates that the composition is used for restoration or augmentation of soft tissue. The article of manufacture may comprise (a) a first container with a composition contained therein, wherein the composition comprises multilayered ADM matrices, e.g. implants; and (b) a second container with a composition contained therein, wherein the composition comprises a further therapeutic agent.

Kits in certain embodiments may further comprise a package insert indicating that the compositions can be used for soft tissue restoration or augmentation. Alternatively, or additionally, the kit may further comprise a second (or third) container comprising a pharmaceutically-acceptable buffer, phosphate-buffered saline, Ringer's solution and dextrose solution. It may further include other materials desirable from a commercial and user standpoint, including other buffers and diluents.

In one aspect, kits are provided that comprise multilayered ADM implants designed for implantation into one or more breasts of a subject. The multilayered ADM implants contained in the kits will have volumetric dimensions that produce specific breast shapes when implanted. In one embodiment, the kit contains one or more multilayered ADM implant sizes of different volumes are provided thereby providing the surgeon with a kit containing an implant size suitable for the needs of the patient.

### EXAMPLE

Postmastectomy breast reconstruction has been steadily increasing in the United States, with a 75% increase since 2000. Implant-based breast reconstruction is the simplest and most common form of breast reconstruction, however, patients with implants may be prone to infection, seroma, rupture, capsular contracture, implant visibility, palpability, and rippling and with limited implant longevity, implants likely necessitate 1 or 2 additional surgeries in a woman's lifetime. A hybrid method for breast reconstructions comprising the steps of (i) removing a sample of flap tissue from the subject; (ii) forming a multilayered ADM composition; and (iii) introduction of the flap and multilayered ADM composition to at least one breast of a subject.

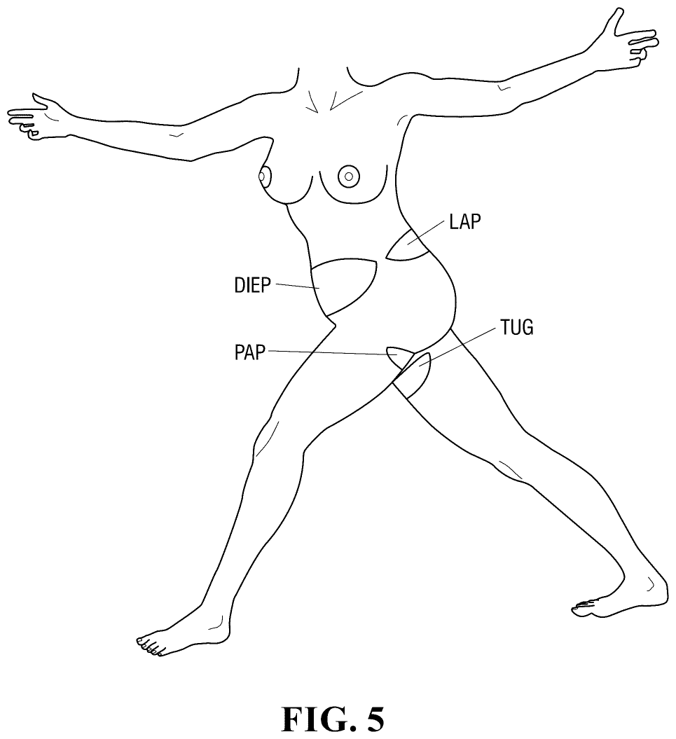

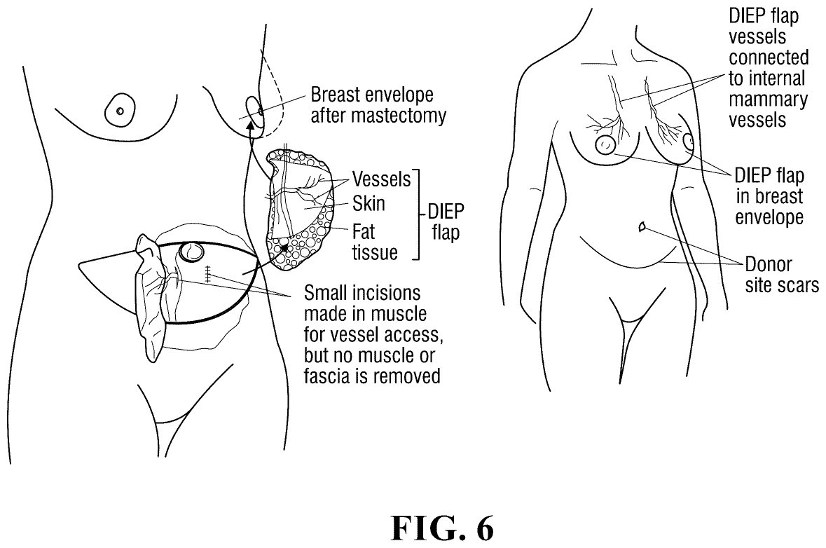

Breast reconstruction using natural tissue is also an option for breast reconstruction. With the use of women's own tissue, the autologous-reconstructed breast has the appearance and feel of a soft natural breast, which responds to changes in body habitus similarly to natural breasts. There are a variety of types of autologous reconstruction methods, with donor tissue originating from different parts of the body (FIG. 5). The most common method of autologous reconstruction is the deep inferior epigastric perforator (DIEP) Flap. In DIEP flap reconstruction, the flap is harvested in an elliptical shape extending across the entire lower abdomen. Small incisions are made in the rectus abdominis muscle for vessel access but no muscle or fascia is removed (FIG. 6). The major disadvantages of DIEP flap reconstruction are the prolonged recovery, limited donor site volume, and donor site complications, such as significant donor scar. Additionally, because the DIEP flap donor site can only be used once, many women choosing DIEP flap breast reconstruction often consider bilateral mastectomies. Alternative donor sites include tissue from the flanks in the lumbar artery perforator flap, the thighs with the profunda artery perforator flap or a transverse upper gracilis (TUG) flap, or the buttocks with the gluteal artery perforator flap.

Although implant-based breast reconstructions typically require less operating time, leave fewer scars, and avoid the risk of donor site morbidity, many patients prefer the permanence, esthetics, and texture of flap-based breast reconstructions. Although there are many benefits to microsurgical flap reconstruction, this is not a viable option for all patients. Namely, patients who desire autologous breast reconstruction but lack adequate flap volume to match the necessary or desired breast volume present a reconstructive challenge. Lacking adequate flap volume can severely compromise the functional and esthetic outcome of breast reconstruction. To address this, alternative reconstructive methods may be considered, including fat grafting of autologous flaps, multilayered flaps, and hybrid breast reconstruction (HBR).

Multilayered flaps are the combination of multiple flaps to reconstruct the breasts. In unilateral reconstruction, the multilayered DIEP is the most commonly used approach, whereby the entire abdominal flap is used to reconstruct one breast. In bilateral breast reconstructions or those situations where the abdominal donor site is not available, alternative donor sites, with or without multilayered flaps, can be used. However, these options are technically more complex, often requiring multiple microvascular anastomoses, longer operative duration, and potentially additional donor-site morbidity.

A solution to the discordance between desired breast reconstruction volume and available donor flap volume is the use of alloplastic and bioprosthetic materials to augment the flap volume in a single-stage hybrid approach. Historically, the latissimus dorsi flap has been used concurrently with an implant. However, this technique requires the sacrifice of a major muscle, which often leads to weakness, reduced mobility, and contour deformity at the donor site. In 2018, Momeni and Kanchwala (Plast Reconstr Surg 2018 November;142(5): 1109-1115) pioneered a new approach that combines a prepectoral implant secured with ADM along and covered with an abdominal free flap in a single-stage procedure. Similar to previous techniques, this allowed for control of both the soft tissue envelope as well as the size and projection of the breast mound. The advantage of this technique lies in the ability to eliminate the downside of submuscular placement (i.e, increased risk of bleeding, pain, and animation deformity) while also reducing the tissue demands on the donor site allowing a more esthetic abdominal closure. Alleviating the need to harvest the entire reconstruction volume at the abdomen allows the donor site scar to be lower, well-hidden, and less tight. There is also greater flexibility as surgeons can select an implant size that best meets patient needs. Esthetically, soft tissue coverage over the implant decreases implant palpability, visibility, and rippling.

Unlike the aforementioned alternative treatment options, the hybrid technique does not significantly increase the duration or complexity of the reconstruction. Other less established yet possible flap options for HBR include the thoracodorsal artery perforator, transverse rectus abdominus musculocutaneous, TUG, and inframammary adipofascial laps. Across all types of HBR, the flap provides vascularized soft tissue coverage to optimize appearance, feel, and minimize alloplastic-related complications. The underlying implant or ADM provides core projection and volume.

Novel methods are disclosed herein for augmenting soft tissue, including preferably at least one breast of a subject, wherein said method combines surgical flap reconstruction with the use of multilayered pre-pectoral acellular dermal matrix (ADM), without the use of an implant, thereby allowing for additional core projection without any implant-associated risks. This method is distinguished from previously disclosed methods by the use of flaps in conjunction with multilayered ADM compositions. Accordingly, the disclosed method specifically comprises the steps of (i) removing a sample of flap tissue from the subject; (ii) forming multilayered ADM; and (iii) introduction of the flap and multilayered ADM compositions to at least one breast of a subject.

The disclosed breast reconstruction technique is an innovative option for women who wish to avoid an implant in their breast reconstruction but lack adequate donor flap site volume to achieve their desired breast volume. The subject to be treated may be a human or more particularly a female human. The method disclosed herein may be used in the reconstruction of the breast mound after lumpectomy, quadrantectomy, mastectomy, and/or radiation therapy. The method may also be used in cosmetic surgery for augmentation of breast size and shape.

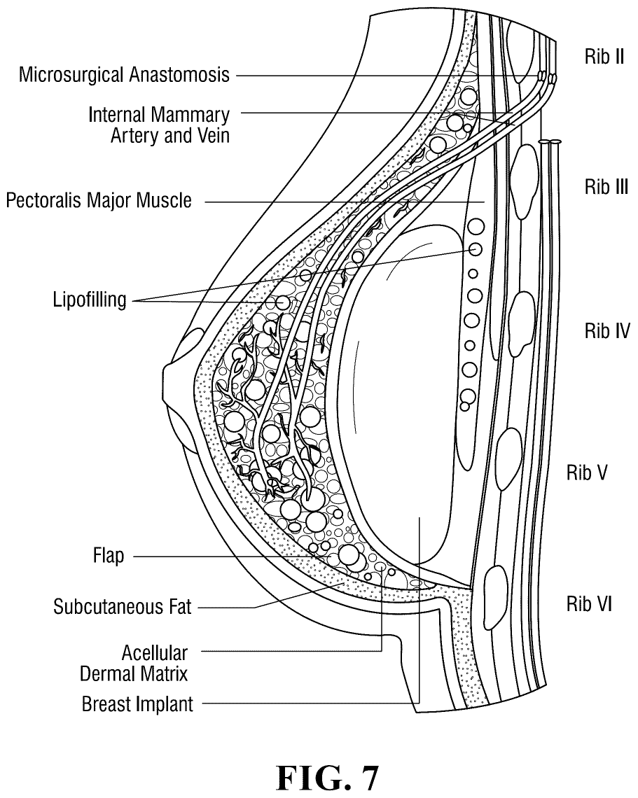

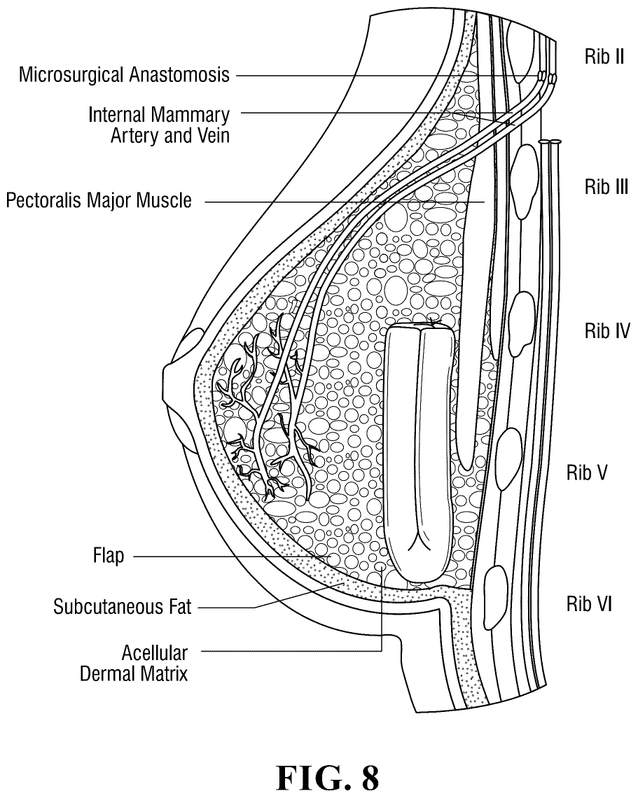

In a specific embodiment, the Hybrid Flap and Prepectoral Acellular Dermal Matrix (HyPAD®) technique disclosed herein combines surgical flap reconstruction with the use of multilayered prepectoral ADM in the place of an implant. An example of the latter is a traditional hybrid breast reconstruction, that encompasses a surgical flap reconstruction with the use of a prepectoral implant (FIG. 7). The multilayered ADM serves the similar purpose as a small implant, but to a lesser degree, providing soft tissue augmentation of the flap and core projection (FIG. 8).

As one step in the disclosed hybrid method, tissue is derived from an area of the body for performance of surgical flap reconstruction. Said flap surgery involves the taking of a section of tissue from one area of the body and relocating it to create a new breast mound. Possible types of flaps for use in the disclosed hybrid method include, for example, (i) a transverse rectus abdominis muscle (TRAM) flap, which includes tissue, including muscle, from your abdomen, (ii) an abdomen deep inferior epigastric perforator (DIEP) flap where only skin and fat are removed leaving most of the abdominal muscle in place, (iii) an abdomen superficial inferior epigastric artery (SIEA) flap which is a variation on the DIEP flap wherein the flap uses the same abdominal tissue as the DIEP flap but relies on blood vessels that aren't as deep within the abdomen, (iv) a back latissimus dorsi flap which takes skin, fat and muscle from the upper back, tunneling it under the skin to the chest, (v) a gluteal flap that takes tissue from the buttocks and transplants it to the chest area, (vi) an inner thigh (TUG) flap that uses muscle and fatty tissue from the bottom of the buttocks to the inner thigh (vii) a lumbar artery perforator (LAP) flap or (viii) an aprofunda artery perforator (PAP) flap. The choice of flap tissue, for use in the hybrid method, will depend upon the patient's amount of available tissue at each of the choice flap sites. In a preferred embodiment, a (DIEP) flap is used. In additional embodiments, the flap sample may be obtained from sources other than the subject. For example, the flap sample may be obtained from another human, a human that is genetically related to the subject, or a human that is a twin of the subject.

When assessing a patient's candidacy for receiving an autologous flap reconstruction, surgeons may consider the volume of the flap available at the donor site relative to the desired breast reconstruction volume. Careful preoperative assessment of a patient's desired breast volume, existing native breast dimensions, and body habitus can help guide the surgeon and patient's expectations of treatment options and esthetic outcome.

In an embodiment, removal of the flaps is affected by methods that minimize trauma and minimize manipulation of the tissue removed from the subject. Methods for flap removal and separation are well known in the medical art. Advantageously, the use of autologous flaps reduces the likelihood of tissue rejection. Autologous tissue breast reconstruction using the tissue derived flaps can be accomplished using one of two surgical methods. The first is referred to as pedicled flap surgery wherein the surgeon cuts some of the blood vessels to the tissue to be transferred but keeps other blood vessels intact. Tunneling the tissue beneath the skin to the chest area, the surgeon then creates the new breast mound or pocket for the implant. In the second surgical method, referred to as free flap surgery, the surgeon disconnects the tissue completely from its blood supply and reattaches the tissue to new blood vessels near the chest.

The placement of the multilayered ADM may be entirely in the prepectoral region, along the vertical meridian at the inframammary fold. This allows for optimal breast projection. Furthermore, the ADM serves as a barrier to protect the flap and vascular pedicle should patients decide to further augment the reconstruction with an implant at a later stage of reconstruction.

In some examples, ADM may be placed under the muscle, for example, as done in a secondary procedure to avoid affecting the flap pedicle previously placed ADM, or in an augmentation setting to avoid mammography interference, or in a radiated breast to avoid potential exposure of the ADM.

The use of the disclosed hybrid approach with concurrent flap transfer and multilayered ADM placement allows for a single-stage procedure that achieves a desirable look and feel while limiting donor site morbidity and postoperative implant-related problems including palpability, rippling, capsular contracture, and reconstructive failures. It is further recognized that the entire procedure comprising the steps of applying the flap and multilayered ADM to at least one breast of a subject, comprising removing a sample of flap tissue from the subject; forming multilayered ADM and introduction into breast of subject can be repeated on one or more breasts of a subject. Repeating the procedure may be desirable as compared to a single procedure in instances where greater degrees of breast enlargement are sought.

In an embodiment, the disclosed hybrid reconstruction procedure can be done on the same day as mastectomy, which is known as “immediate reconstruction.” For women with a previous mastectomy, the method may be performed at a separate time after a mastectomy. This is known as “delayed reconstruction.” Almost all women are candidates for immediate reconstruction, so speaking with your team prior to mastectomy about reconstruction is important. Occasionally, a “bridging implant” reconstruction may be used before breast reconstruction to allow women with a more advanced disease requiring radiation treatment to benefit from skin sparing or nipple sparing mastectomy as well.

In an embodiment, to improve the aesthetic outcomes 3D virtual surgical planning may be used. 3D photography will allow assessment of the breast shape, symmetry and volume. Flap site MRI or CT scans will also allow better visualization of the vascular anatomy and guide the surgeon to potential flap donor site volume and tissue thickness as to which approach will lead to the most optimal result.

As disclosed below, the use of ADM during autologous breast reconstruction provides patients with an alternative to implants that allows them to obtain their desired breast volume. Given the aesthetic impact that ADM has on the total amount of flap weight and projection, it is recommended that the HyPAD® technique be considered for patients who desire a breast that cannot be restored with their own flap weight and wish to avoid implants.

Consecutive patients who underwent the HyPAD® technique during the study were identified. All patients lacked adequate donor site volume and wished to avoid the placement of implants during their reconstruction. Demographic information and outcomes were assessed. Intraoperatively, the weight (grams, g) of the mastectomy specimen, flap donor site, and stacked ADM were recorded.

A single, cadaveric, ADM is used per breast, with the thickness of the ADM selected based on the volume necessary. The ADM is sterilely removed from its box and rinsed with antibiotic solution. The ADM is cut into two pieces, a square and a thin rectangular piece. The thin rectangular piece is then folded onto itself, resulting in a smaller square ADM construct. This smaller ADM square construct is then then placed in the middle of the larger square of ADM. Opposite corners of the larger square are then sutured to each other and the smaller square, creating a square-shaped ADM construct in which the larger ADM square completely enveloped the smaller ADM square. All sutures are placed with a monofilament 3-0 Polydioxanonesuture (PDS) suture. The resulting ADM construct is now placed in the pre-pectoral plane, posterior to the flap.

Demographic information and outcomes were assessed. Intraoperatively, the weight (grams, g) of the mastectomy specimen, flap donor site, and stacked ADM were recorded. Complications were also reviewed for study subjects. Follow up was at least 1 year for all patients. Statistical analysis was performed using the Data Analysis ToolPak in Excel.

During the study period, 21 patients (n=21) were identified. The mean age of patients at the time of surgery was 48.9±7.85 years. The mean body mass index (BMI) was 24.1 kg/m²±3.3 kg/m². The mean mastectomy specimen weight was 436.35 g, and the mean flap weight was 370.73 g, posing an average discrepancy of 15.04% or 65.62 g. The average weight of the ADM used to augment the flap was 81.76 g. This weight accounted for an average of 18.07% of the total reconstructed breast weight. The use of ADM during autologous breast reconstruction provides patients with an alternative to implants that allows them to obtain their desired breast volume. Given the aesthetic impact that ADM has on the total amount of flap weight and projection, it is recommended that the HyPAD® technique be considered for patients who desire a breast that cannot be restored with their own flap weight and wish to avoid implants.

During the study period, twenty-one (21) who underwent the HyPAD® technique were identified. The mean age of patients at the time of surgery was 48.9±7.85 years (range 42-57). The mean body mass index (BMI) was 24.1 kg/m²±3.3 kg/m². The mean mastectomy specimen weight was 436.35 g and the mean flap weight was 370.73 g, posing an average discrepancy of 15.04%. The average weight of the ADM used to augment the flap was 81.76 g. This weight accounted for an average of 18.07% of the total reconstructed breast weight. There were no complications including infection, seroma, mastectomy skin flap necrosis, red breast syndrome, and/or need for ADM removal.

What is claimed:

1. A pharmaceutical composition comprising multilayered acellular matrices wherein the multilayered acellular matrices comprises two or more layers.

2. The pharmaceutical composition of claim 1, wherein the multilayered acellular matrices are multilayered acellular dermal matrices (ADMs).

3. The pharmaceutical composition of claim 1, wherein the multilayered acellular matrices is derived from an animal source.

4. The pharmaceutical composition of claim 1, wherein a sheet of acellular matrices is folded on itself at least once.

5. The pharmaceutical composition of claim 1, wherein two or more sheets of acellular matrices are layered on top of each other.

6. The pharmaceutical composition of claim 1, wherein the multilayered acellular matrices are in the shape of a mound.

7. The pharmaceutical composition of claim 1, wherein the multilayered acellular matrices further comprises a therapeutic agent, a peptide or a nucleic acid.

8. A pharmaceutical composition comprising multilayered acellular matrices, for enhancing the volume of soft tissue in a subject undergoing a surgical procedure, wherein the multilayered ADM is in an amount effective to provide for restoration or augmentation of soft tissue shape when administered to the subject.

9. The application of the composition of claim 1, in preparing products for repairing or filling soft tissues.

10. A method for augmenting soft tissue in a subject having inadequate soft tissue volume in a selected anatomic site comprising: identifying an anatomic site in a subject within a soft tissue in need of augmentation; selecting a multilayered acellular matrices composition; implanting the pharmaceutical composition of claim 1 in or proximate to the soft tissue; and securing at least a portion of the multilayered composition in or near the soft tissue.

11. A method for restoring a soft tissue loss comprising: identifying an anatomic site in a subject within a soft tissue in need of restoration; selecting a multilayered acellular matrices composition; implanting the pharmaceutical composition of claim 1 in or proximate to the soft tissue; and securing at least a portion of the multilayered composition in or near the soft tissue.

12. The method of claim 11, wherein the subject has experienced soft tissue losses from surgery, tumor removal, trauma, aging, or congenital malformation.

13. The method of claim 10, wherein the soft tissue is tissue is breast, facial, trunk, extremity, abdominal, ear, nose or penile soft tissue.

14. The method of claim 11, wherein the soft tissue is tissue is breast, facial, trunk, extremity, abdominal, car, nose or penile soft tissue.

15. A kit comprising the pharmaceutical composition of claim 1 and instructions for their use.

16. A method of implanting the multilayered acellular matrice of claim 1 comprising at least the steps of: (i) making at least one incision to access the patient's breast tissue, (ii) creating a plane to receive the multilayered acellular matrice implant below the breast mount (subglandular) or below the pectoralis chest muscle (subpectoral) (iii) placing the multilayered acellular matrice implant in the respective plane, and (iv) securing the multilayered acellular matrice implant to the tissue surrounding and (v) closing the incisions in the breast. 17. A hybrid method for breast reconstructions comprising the steps of (i) removing a sample of flap tissue from the subject; (ii) forming the pharmaceutical composition of claim 1; and (iii) introduction of the flap and pharmaceutical composition of claim 1 to at least one breast of a subject.

17. The hybrid method of claim 16, wherein the flap is an autologous flap.

18. The hybrid method of claim 16, wherein the flap tissue is selected from the group consisting of, a transverse rectus abdominis muscle (TRAM) flap; an abdomen deep inferior epigastric perforator (DIEP) flap; an abdomen superficial inferior epigastric artery (SIEA) flap; a back latissimus dorsi flap; a gluteal flap; an inner thigh (TUG) flap; a lumbar artery perforator (LAP) flap and an aprofunda artery perforator (PAP) flap.

19. The hybrid method of claim 16, wherein the subject is a woman who has undergone a lumpectomy, quadrantectomy, total or partial mastectomy, and/or radiation therapy.
